(19) United States
(12) Patent Application Publication
(10) Pub. No.: US 2026/0319569 A1
(43) Pub. Date: Oct. 15, 2026

(54) METHODS OF AND SYSTEMS FOR ESTIMATING A TOPOGRAPHY OF AT LEAST TWO PARTS OF A BODY

(71) Applicant: TEXAVIE TECHNOLOGIES INC., Vancouver (CA)

(72) Inventors: Peyman Servati, Vancouver (CA); Amir Servati, Coquitlam (CA); Zenan Jiang, Richmond (CA); Saeid Soltanian, North Vancouver (CA); Harishkumar Narayana, Vancouver (CA); Jennifer Angelica Ongko, Vancouver (CA)

(73) Assignee: TEXAVIE TECHNOLOGIES INC., Vancouver (CA)

(21) Appl. No.: 19/381,506

(22) Filed: Nov. 6, 2025

Related U.S. Application Data
(63) Continuation of application No. 17/048,863, filed on Apr. 18, 2019, now abandoned.
(60) Provisional application No. 62/660,168, filed on Apr. 19, 2018.

(51) Int. Cl. G06F 3/01; A61B 5/00; A61B 5/107; A61B 5/11

(52) U.S. Cl. CPC G06F 3/011; A61B 5/1077; A61B 5/1114; A61B 5/6804; G06F 3/017; A61B 2503/12

(57) ABSTRACT

A method of estimating a topography of at least first and second parts of a body may involve: causing at least one processor circuit to receive at least one signal representing at least one measurement of deformation of at least a portion of the body; causing the at least one processor circuit to associate the deformation with relative positions of at least the first and second parts of the body; and causing the at least one processor circuit to produce at least one output signal representing the relative positions of at least the first and second parts of the body. Systems are also disclosed.

### CROSS-REFERENCES TO RELATED APPLICATIONS

This application is a continuation of U.S. patent application Ser. No. 17/048,863, filed Oct. 19, 2020, which is a National Stage Entry of International Patent Application No. PCT/CA2019/050493, filed Apr. 18, 2019, which claims the benefit of U.S. Provisional Patent Application No. 62/660,168, filed Apr. 19, 2018, each of which applications is incorporated by reference herein in its entirety.

### FIELD

This disclosure relates generally to methods of and systems for estimating a topography of at least two parts of a body.

### RELATED ART

Some applications may involve monitoring a topography of parts of a body. However, some methods of monitoring a topography of parts of a body may require high power consumption, have a limited field of view, may be uncomfortable to wear, may have low accuracy, or may depend on complex algorithms.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to at least one embodiment, there is disclosed a method of estimating a topography of at least first and second parts of a body, the method comprising: causing at least one processor circuit to receive at least one signal representing at least one measurement of deformation of at least a portion of the body; causing the at least one processor circuit to associate the deformation with relative positions of at least the first and second parts of the body; and causing the at least one processor circuit to produce at least one output signal representing the relative positions of at least the first and second parts of the body.

According to at least one embodiment, there is disclosed a system for estimating a topography of at least first and second parts of a body, the system comprising: a means for receiving at least one signal representing at least one measurement of deformation of at least a portion of the body; a means for associating the deformation with relative positions of at least the first and second parts of the body; and a means for producing at least one output signal representing the relative positions of at least the first and second parts of the body.

According to at least one embodiment, there is disclosed a system for estimating a topography of at least first and second parts of a body, the system comprising at least one processor circuit configured to, at least: receive at least one signal representing at least one measurement of deformation of at least a portion of the body; associate the deformation with relative positions of at least the first and second parts of the body; and produce at least one output signal representing the relative positions of at least the first and second parts of the body.

Other aspects and features will become apparent to those ordinarily skilled in the art upon review of the following description of illustrative embodiments in conjunction with the accompanying figures.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a perspective view of a system for estimating a topography of at least two parts of a body according to one embodiment.

FIG. 2 is a perspective view of a sensor of the system of FIG. 1.

FIG. 3 is a perspective view of a deformation sensor of the sensor of FIG. 2.

FIG. 4 is an enlarged view of the deformation sensor of FIG. 3.

FIG. 5 is a perspective view of a deformation sensor according to another embodiment.

FIG. 6 is a schematic illustration of a processor circuit of a computing device of the system of FIG. 1.

FIG. 7 is a schematic illustration of program codes in a program memory of the processor circuit of FIG. 6.

FIG. 8 is a schematic illustration of an example of one or more measurements of deformation by the sensor of FIG. 2 when fingers of a hand on a forearm are in an open position.

FIG. 9 is a schematic illustration of another example of one or more measurements of deformation by the sensor of FIG. 2 when the fingers are positioned in a fist.

FIG. 10 is a schematic illustration of another example of one or more measurements of deformation by the sensor of FIG. 2 when an index finger of the hand is in a pointing position.

FIG. 11 is a schematic illustration of body parts of a musculoskeletal model stored in a storage memory of the processor circuit of FIG. 6.

FIGS. 12 and 13 are other schematic illustrations of body parts of the musculoskeletal model stored in the storage memory of the processor circuit of FIG. 6.

FIG. 14 is a schematic illustration of a sequence of anatomical positions of the hand.

FIGS. 15-17 illustrate a sensor according to another embodiment.

FIGS. 18 and 19 illustrate a sensor according to another embodiment.

FIG. 20 is a perspective view of a system for estimating a topography of at least two parts of a body according to another embodiment.

FIG. 21 is a perspective view of a sensor according to another embodiment.

FIG. 22 is a perspective view of a sensor according to another embodiment.

### DETAILED DESCRIPTION

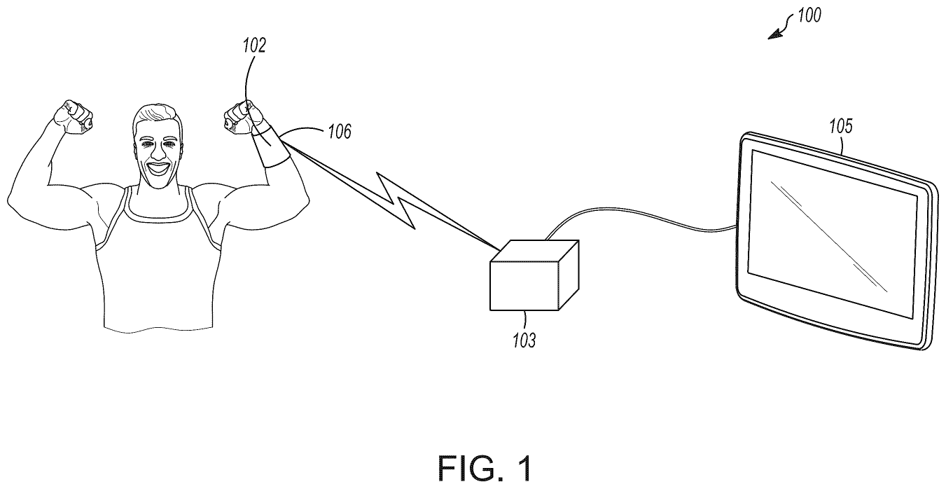

Referring to FIG. 1, a system for estimating a topography of at least two parts of a body is shown generally at 100 and includes a sensor 102, a computing device 103, and a display device 105. In general, “body” herein may refer to a human body, to a non-human animal body, or to another body.

### Display Device

In the embodiment shown, the display device 105 is a television screen. However, display devices of alternative embodiments may vary. For example, a display device of an alternative embodiment may be a virtual-reality goggle, an augmented-reality goggle, a mixed-reality goggle, a mobile phone, a smartphone, a tablet, a projected image on a screen, or any display device of a visual interactive system.

### Sensor

Referring to FIG. 2, the sensor 102 includes a resiliently deformable material 104. Such a resiliently deformable material may include one or more materials such as spandex, soft rubber, silicone, natural fibers, polymers, cotton, nylon, other yarns, fabric, smart textile, clothing, or other related textiles, which may be breathable or otherwise chosen for comfort or other reasons. Further, one or more materials of the sensor 102 may be chosen such that textile fabric structure, fiber composition, mechanical properties, hand properties, comfort properties, proper direction for sensor placement, or other factors may facilitate accurate measurements such as those described herein, for example.

Further, the resiliently deformable material 104 is sized to be received tightly on (or conform to) a forearm 106 of a body, and configured to surround the forearm 106. The sensor 102 may therefore be referred to as a sensor textile. The sensor 102 includes a plurality of deformation sensors, such as deformation sensors 108 and 110, for example. When the sensor 102 is worn on the forearm 106, the deformation sensors of the sensor 102 are positioned against an external surface of the forearm 106 and positioned to measure deformations of the forearm 106 that may be caused by movement of muscles, bones, tendons, or other tissues in the forearm 106.

In the embodiment shown, the deformation sensors of the sensor 102 are positioned in the sensor 102 in a two-dimensional array including a row of deformation sensors shown generally at 112, a row of deformation sensors shown generally at 114, a row of deformation sensors shown generally at 116, and a row of deformation sensors shown generally at 118. The rows of deformation sensors 112, 114, 116, and 118 are spaced apart from each other such that, when the sensor 102 is worn on the forearm 106, the rows of deformation sensors 112, 114, 116, and 118 are spaced apart from each other in a direction along the forearm 106, and each of the rows of deformation sensors 112, 114, 116, and 118 includes a plurality of deformation sensors spaced apart from each other in an anterior-posterior direction when worn on the forearm 106. Therefore, the deformation sensors of the sensor 102 are spaced apart from each other in at least two directions and therefore form a grid or two-dimensional array. The sensor 102 is an example only, and alternative embodiments may differ. For example, in alternative embodiments, deformation sensors may be positioned in other ways, such as an irregular pattern over two dimensions that may correspond to anatomical features. For example, to detect radial artery pulsations, a high-density array of sensors can be placed close to a radial artery and other sensors on the forearm for movement detection.

The sensor 102 also includes a data processing unit 120 in communication with the deformation sensors of the sensor 102. Each of the rows of deformation sensors may include a respective plurality of stretchable wire lines, such as the stretchable wire line 122 shown in the row of deformation sensors 112, and a stretchable bus line 124 may connect the stretchable wire lines (such as the stretchable wire line 122, for example) to the data processing unit 120.

In the embodiment shown, the data processing unit 120 is configured to communicate wirelessly with the computing device 103, for example according to a Bluetooth™, WiFi, Zigbee™, near-field communication (“NFC”), or 5G protocol, or according to another protocol for wireless communication. However, in alternative embodiments, the data processing unit 120 may communicate with the computing device 103 using one or more wires or in other ways. Additionally, the data processing unit 120 may implement functions including but not limited to analog signal conditioning and amplification, analog to digital conversion, signal filtering and processing, signal classification and recognition, machine learning, and wireless data transfer. The data processing unit 120 may also include battery and storage devices or wireless charging or other energy harvesting components such as energy generation from movement or environmental light, for example.

In general, information (such as information representing measurements of deformations by the sensor 102, for example) may be transferred wirelessly or otherwise to the computing device 103 in real time. Alternatively, such information can be stored in the data processing unit 120 or elsewhere, and transferred to the computing device 103 at a later time.

Further, a communication rate between the processing unit 120 and the computing device 103 may be about a few megabytes per second, about a few thousand bytes per second, about a few bytes per second, about a few bytes every hour, or about a few bytes every day, depending for example on energy-usage requirements or accuracy or refresh rates of data that may be needed for a specific application. Such a communication rate may, for example, be high in gaming and sports applications and may be much lower in other applications. Such a communication rate can be adaptively modified to save energy, for example increasing when demand is high and decreasing when there is little or no need for data.

The data processing unit 120 may also include one or more inertial measurement units (“IMUs”) such as one or more accelerometers, one or more gyroscopes, one or more magnetometers, or a combination of two or more thereof, which may detect orientation and angles of movement as spatial reference point for tissue, for example. The processing unit 120 may fuse measurements of deformation (or topography data) with data from one or more such IMUs, which may improve accuracy and functionality. The data processing unit 120 may also include one or more global positioning system (GPS) capabilities (or one or more other locating devices), which may facilitate identifying one or more locations of the sensor 102 or long-range movements of the sensor 102.

The data processing unit 120 or the sensor 102 may also include one or more haptic devices, or other devices which may apply tactile or other feedback to a person wearing the sensor 102.

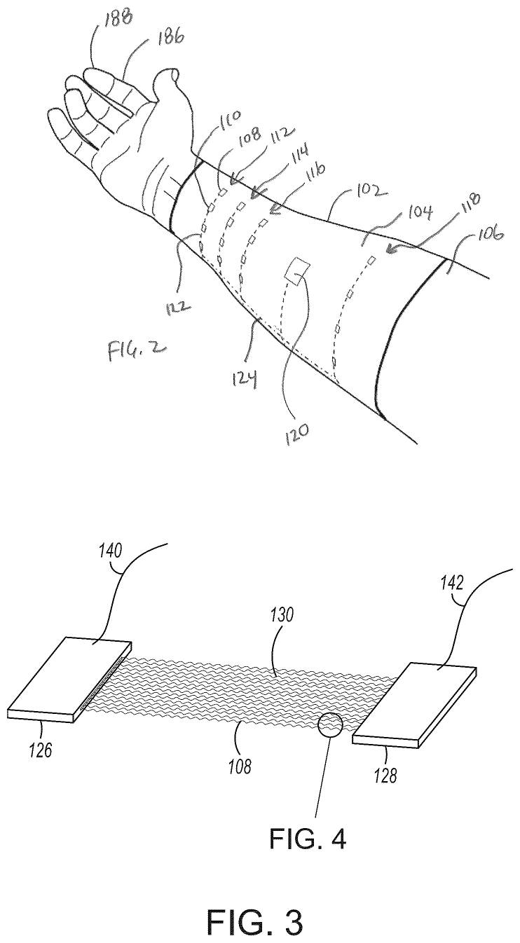

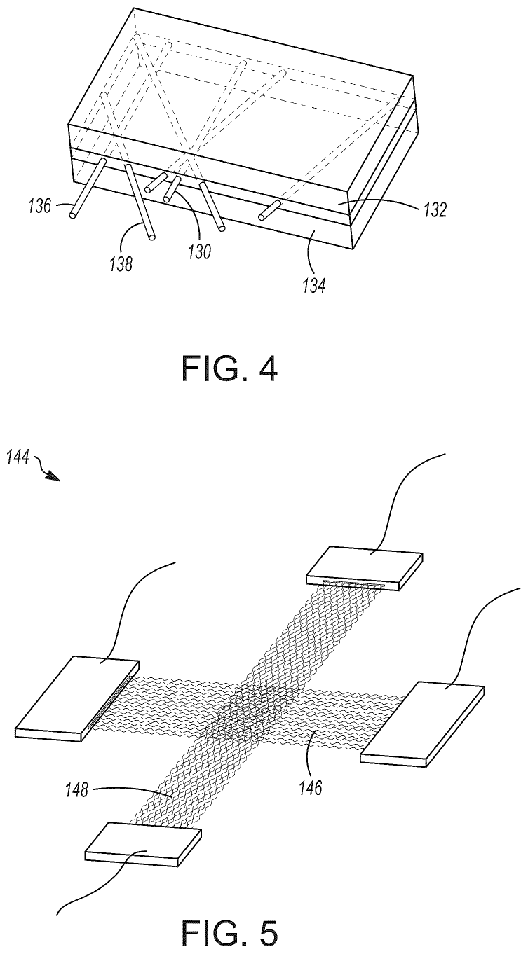

Deformation sensors such as those described herein may be similar to sensors that are described in U.S. Pat. No. 9,494,474 . For example, referring to FIG. 3, the deformation sensor 108 is shown in greater detail and includes an electrode 126, an electrode 128, and a fiber mesh 130 extending between and in electrically conductive contact with the electrodes 126 and 128. Referring to FIG. 4, the deformation sensor 108 also includes resiliently deformable encapsulating films 132 and 134 encapsulating the fiber mesh 130. As shown in FIG. 4, the fiber mesh 130 includes a plurality of elongate fibers, such as fibers 136 and 138, for example, with each including an electrical conductor having an electrically conductive exterior surface. As also shown in FIG. 3, an electrical lead 140 may be in electrically conductive contact with the electrode 126, and an electrical lead 142 may be in electrically conductive contact with the electrical lead 128, so that electrical resistance of the fiber mesh 130 may be measured. As described in U.S. Pat. No. 9,494,474 for example, electrical resistance of the fiber mesh 130 may indicate strain or deformation of the fiber mesh 130.

Referring to FIG. 5, a deformation sensor according to another embodiment is shown generally at 144 and includes a deformation sensor 146 and a deformation sensor 148. The deformation sensors 146 and 148 may be similar to the deformation sensor 108 as described above, although the deformation sensors 146 and 148 may be positioned generally perpendicular relative to each other, and may function together as a deformation sensor.

The deformation sensors described above are examples only, and alternative embodiments may differ. For example, deformation sensors according to other embodiments may include one or more carbon-black-based force-sensitive and strain-sensitive sensors, one or more capacitive deformation sensors, one or more other types of force or deformation sensors, a combination of two or more thereof, or other methods to extract deformation and location of the topography of the body.

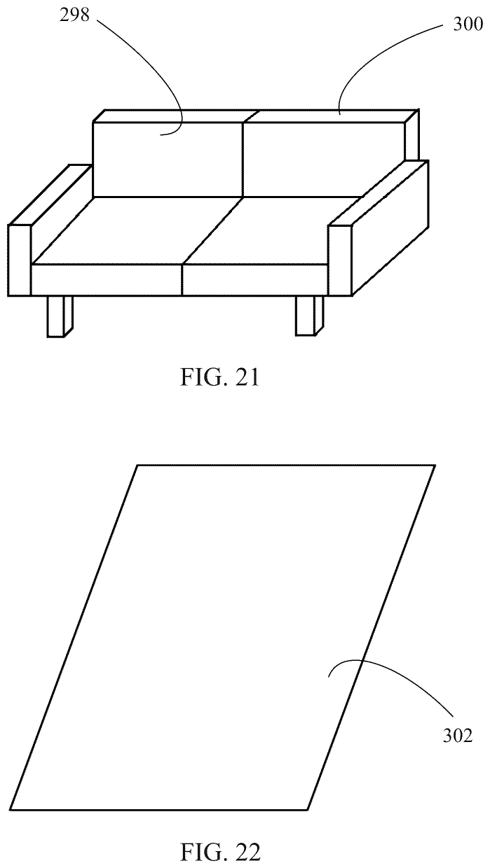

The sensor 102 is an example only, and sensors of alternative embodiments may differ. For example, a sensor of an alternative embodiment may not be worn on a body, and such as sensor may be a furniture cover 298 of furniture 300 (as shown in FIG. 21) or bedding 302 (as shown in FIG. 22), for example.

Further, the embodiment shown includes one sensor 102, but alternative embodiments may include more than one sensor on one body or (as shown in FIG. 20, for example) on more than one body. As also shown in FIG. 20, such multiple sensors may be in communication with each other using one or more computing networks.

### Computing Device

In general, the computing device 103 may include a personal computer, a laptop, a tablet, a stand-alone computing device, or any computing hardware for a virtual-reality goggle, an augmented-reality goggle, a mixed-reality goggle, a mobile phone, a smartphone, a television screen, a gaming device, a projector for projecting images on a screen, or any display device of a visual interactive system.

Also, although FIG. 1 illustrates the sensor 102 separate from the computing device 103, and the computing device 103 separate from the display device 105, the sensor 102 may be combined with the computing device 103 in some embodiments, or the computing device 103 may be combined with the display device 105 in some embodiments. Still other embodiments may include one or more different elements that may be separated or that may be combined in different ways.

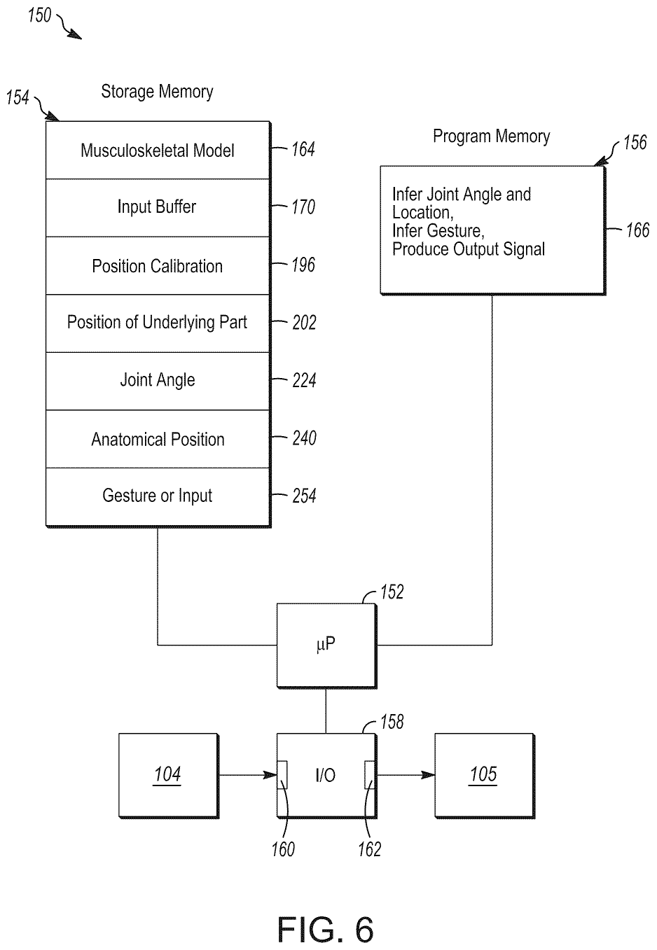

Referring to FIG. 6, the computing device 103 includes a processor circuit shown generally at 150 which includes a microprocessor 152. The processor circuit 150 also includes a storage memory 154, a program memory 156, and an input/output (“I/O”) module 158, all in communication with the microprocessor 152.

In general, the storage memory 154 includes stores for storing storage codes as described herein, for example. In general, the program memory 156 stores program codes that, when executed by the microprocessor 152, cause the processor circuit 150 to implement functions of the computing device 103 such as those described herein, for example. The storage memory 154 and the program memory 156 may be implemented in one or more of the same or different computer-readable storage media, which in various embodiments may include one or more of a read-only memory (“ROM”), a random access memory (“RAM”), a hard disc drive (“HDD”), a solid-state drive (“SSD”), a remote memory such as one or more cloud or edge cloud storage devices, and other computer-readable and/or computer-writable storage media.

The I/O module 158 may include various signal interfaces, analog-to-digital converters (“ADCs”), receivers, transmitters, and/or other circuitry to receive, produce, and transmit signals as described herein, for example. In the embodiment shown, the I/O module 158 includes an input signal interface 160 for receiving signals (for example according to one or more protocols such as those described above) from the data processing unit 120 of the sensor 102, and an output signal interface 162 for producing one or more output signals and for transmitting the one or more output signals to the display 105 to control the display 105.

The I/O module 158 is an example only and may differ in alternative embodiments. For example, alternative embodiments may include more, fewer, or different interfaces. Further, the I/O module 158 may connect the computing device 103 to a computer network (such as an internet cloud or edge cloud, for example), and such a computer network may facilitate real-time communication with other computing devices. Such other computing devices may interact with the computing device 103 to permit remote interaction, for example.

More generally, the processor circuit 150 is an example only, and alternative embodiments may differ. For example, in alternative embodiments, the computing device 103 may include different hardware, different software, or both. Such different hardware may include more than one microprocessor, one or more alternatives to the microprocessor 152, discrete logic circuits, or an application-specific integrated circuit (“ASIC”), or a combination of one or more thereof, for example. As a further example, in alternative embodiments, some or all of the storage memory 154, of the program memory 156, or both may be cloud storage or still other storage.

The storage memory 154 includes a musculoskeletal model store 164, which stores codes representing one or more musculoskeletal models of a body. For example, such a musculoskeletal model may represent bones, muscles (such as the flexor digitorum superficialis muscle bundles, for example), tendons, fascia, arteries, and other tissues, including representations of how positions of muscles or other tissues (and movements, contractions and rotations thereof) may be associated with relative positions of body parts, or with angles of flexion, extension, or rotations of joints of the body. In some embodiments, the deformation sensors of the sensor 102 may be positioned to measure deformation of particularly important body parts of the musculoskeletal model.

### Program Memory

In general, the program memory 156 may include program codes that, when executed by the microprocessor 152, cause the processor circuit 150 to implement machine learning or artificial intelligence algorithms such as deep neural networks, deep learning, or support vector machines, for example. Further, the program memory 156 may cause the processor circuit 150 to implement cloud virtual machines.

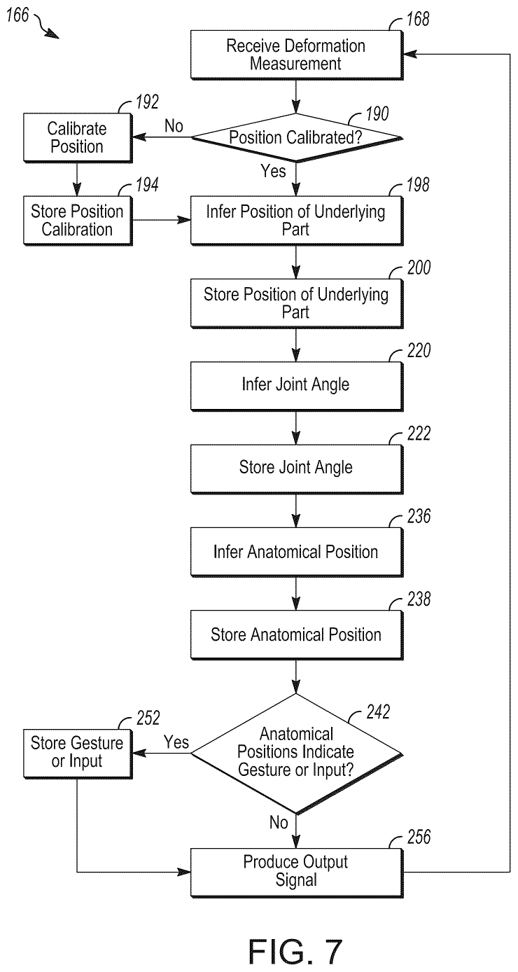

The program memory 156 includes program codes 166, which are illustrated schematically in FIG. 7. Referring to FIGS. 6 and 7, the program codes 166 begin at block 168, which includes codes that, when executed by the microprocessor 152, cause the processor circuit 150 to receive, at the input signal interface 160, one or more signals representing one or more measurements by the sensor 102 of deformation of at least a portion of the forearm 106, and to store codes representing the one or more measurements of deformation in an input buffer 170 in the storage memory 154.

FIG. 8 is a schematic illustration of an example of one or more measurements of deformation that may be represented by codes in the input buffer 170. FIG. 8 illustrates a topography including a plurality of rows such as rows shown generally at 172 and 174, and a plurality of columns such as columns shown generally at 176, 180, 182, and 184. Referring to FIGS. 2 and 8, in the embodiment shown, deformation measurements measured by the deformation sensor 108 may be illustrated in the row 172 and in the column 176 in FIG. 8. Likewise, deformation measurements by other deformation sensors aligned with the deformation sensor 108 but in other rows (such as the rows 114, 116, and 118, for example) may be illustrated in FIG. 8 in the row 172 but in other columns (such as the columns 180, 182, and 184 for deformation sensors in the rows 114, 116, and 118 respectively, for example). Likewise, deformation measurements measured by the deformation sensor 110 may be illustrated in the row 174 and in the column 176 in FIG. 8, deformation measurements of deformation sensors in the row 114 may be illustrated in the column 180, deformation measurements of deformation sensors in the row 116 may be shown in the column 182, and deformation measurements by deformation sensors in the row 118 may be illustrated in the column 184. In other words, FIG. 8 illustrates a topography corresponding to deformation measurements at locations on at least a portion of the forearm 106 as measured by respective deformation sensors at such locations on the forearm 106.

FIG. 8 illustrates deformation measurements according to one embodiment when fingers on a hand 186 on the forearm 106 are open. FIG. 9 illustrates deformation measurements on the forearm 106 when the fingers of the hand 186 are positioned in a fist. FIG. 10 illustrates deformations of the forearm 106 when an index finger 188 of the hand 186 is in a pointing position.

The deformation measurements measured by the deformation sensor 110, may, for example, represent a moving tissue dynamic topography (MTDT) map, which may provide relative changes (in percentage, for example) in one or more signals produced by the deformation sensors at different locations on the forearm 106. The topography examples shown in FIGS. 8-10 are for MTDT sensed from an elbow to a wrist of the forearm 106 and on anterior (or flexor) and posterior (or extensor) sides of the forearm 106. The topography examples shown in FIGS. 8-10 may be measured by the deformation sensors in this embodiment.

Referring back to FIGS. 2 and 6, the musculoskeletal model represented by codes in the musculoskeletal model store 164 may include anatomical features, and the deformation sensors of the sensor 102 may, over time, have varying positions relative to such anatomical features. Therefore, in general, positions of the deformation sensors of the sensor 102 may be calibrated to positions of anatomical features in the musculoskeletal model represented by codes in the musculoskeletal model store 164. Therefore, referring back to FIGS. 6 and 7, after block 168 the program codes 166 may continue at block 190, which includes codes that, when executed by the microprocessor 152, cause the processor circuit 150 to determine whether positions of the deformation sensors of the sensor 102 are calibrated relative to anatomical features of the musculoskeletal model. If not, then the program codes 166 continue at block 192, which includes codes that, when executed by the microprocessor 152, cause the processor circuit 150 to calibrate positions of the deformation sensors relative to the anatomical features. After block 192, the program codes 166 continue at block 194, which includes codes that, when executed by the microprocessor 152, cause the processor circuit 150 to store, in a position calibration store 196 in the storage memory 154, codes representing the position calibration. In general, codes representing such position calibration can be retrieved or corrected from calibration data that may be previously stored in the sensor 102, in the position calibration store 196, elsewhere in the processor circuit 150, in cloud storage, or elsewhere.

After block 194, or if at block 190 the positions of the deformation sensors are calibrated relative to the anatomical features, the program codes 166 continue at block 198, which includes codes that, when executed by the microprocessor 152, cause the processor circuit 150 to infer, according to the deformation measurement as received at block 168 and as stored in the input buffer 170, positions of one or more body parts underlying the deformation sensors of the sensor 102. In general, such underlying body parts may include one or more muscles, one or more bones, one or more tendons, one or more other body parts, or a combination of two or more thereof. The codes at block 198 may involve a statistical learning algorithm trained to associate deformation of a portion of the body with positions of one or more muscles. The program codes 166 then continue at block 200, which includes codes that, when executed by the microprocessor 152 cause the processor circuit 150 to store codes representing the inferred muscle positions in an underlying body part position store 202 in the storage memory 154. Such information regarding such a body part may be stored in the storage memory 154, in cloud storage, or elsewhere for later retrieval. Such information regarding such a body part may indicate, for example, size or activity of a muscle, form or fitness of a muscle, size of the body part, the fit and stretch of the sensor around the body part, or a combination of two or more thereof, for example.

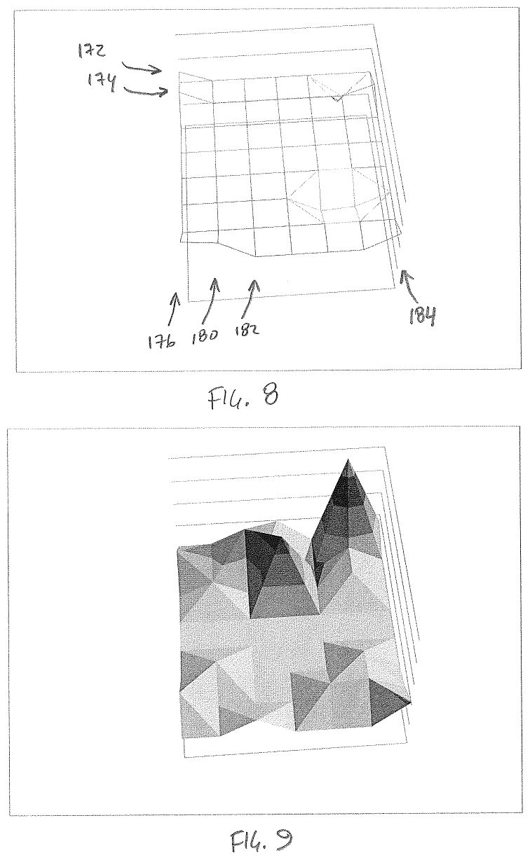

Referring to FIG. 11, the anatomical model may include a model representation of a first anterior muscle 204, a model representation of a second anterior muscle 206, and a model representation of a posterior muscle 208 in the forearm 106. The anterior muscle 204 may be movable in a direction 210, the anterior muscle 206 may be movable in a direction 212, and the posterior muscle 208 may be movable in a direction 214. Measurements of deformation of the forearm 106 by deformation sensors of the sensor 102 may indicate positions of muscles such as the muscles 204, 206, and 208 along their respective directions of movement 210, 212, and 214, for example, and the codes at block 198 may infer respective positions of such muscles along such directions of movement.

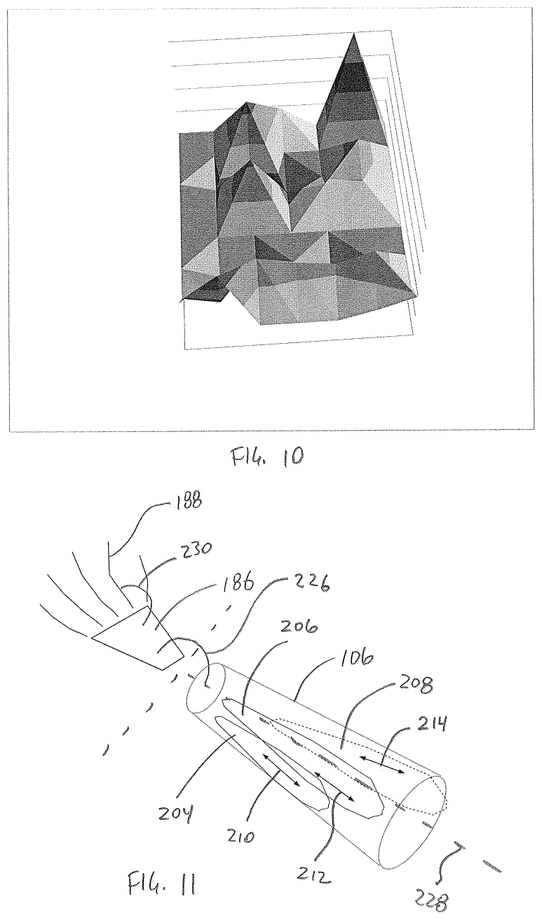

As another example, referring to FIGS. 12 and 13, the forearm 106 includes an ulna bone 216 and a radius bone 218. Rotation of the ulna bone 216 and of the radius bone 218 from the positions shown in FIG. 12 to the positions shown in FIG. 13 causes deformation of the forearm 106 and measurements of such deformation indicate such movement of the ulna bone 216 and of the radius bone 218. The codes at block 198 may infer such positions of the ulna bone 216 and of the radius bone 218 from such deformations of the forearm 106.

Referring back to FIGS. 6 and 7, after block 200, the program codes 166 may continue at block 220, which includes codes that, when executed by the microprocessor 152, cause the processor circuit 150 to infer one or more joint angles from the positions of underlying parts stored in the position of underlying body part store 202. In some embodiments, for example, the codes at block 220 may associate positions of particular muscle bundles (such as flexor carpi radialis, flexor digitorum superficialis, or extensor digitorum, for example) with angles between of one or more bones of the forearm 106, of the hand 186, of fingers of the hand 186, of an elbow adjacent the forearm 106, or of a shoulder of a same arm as the forearm 106. The program codes 166 continue at block 222, which includes codes that, when executed by the microprocessor 152, cause the processor circuit 150 to store, in a joint angles store 224 in the storage memory 154, codes representing one or more joint angles inferred at block 220. Referring to FIG. 11, for example, the codes at block 220 may cause the processor circuit 150 to infer an angle 226 between the hand 186 and a longitudinal axis 228 of the forearm 106. As another example, the codes at block 220 may cause the processor 150 to infer an angle 230 between the hand 186 and the index finger 188. As another example, referring to FIGS. 12 and 13, the codes at block 220 may cause the processor circuit 150 to infer an angle 232 from a reference plane 234.

As the embodiment shown illustrates, embodiments such as those described herein may infer, from deformation of one part of a body (the forearm 106 in the embodiment shown), one or more joint angles between a first part of the body (the forearm 106 in the embodiment shown) where deformation is measured and a second part of the body (such as the hand 186 or one or more fingers of the hand 186) that is not within a sensor (the sensor 102 in the embodiment shown) but that rather may be outside of (or spaced apart from) a part of the body (the forearm 106 in the embodiment shown) where deformation is measured and that may be movable relative to the part of the body where deformation is measured.

Referring back to FIGS. 6 and 7, after block 222, the program codes 166 may continue at block 236, which includes codes that, when executed by the microprocessor 152, cause the processor circuit 150 to infer one or more anatomical positions (or poses) from the one or more joint angles stored in the joint angles store 224. The program codes 166 continue at block 238, which includes codes that, when executed by the microprocessor 152, cause the processor circuit 150 to store, in an anatomical positions store 240 in the storage memory 154, codes representing one or more anatomical positions inferred at block 236. Such anatomical positions or poses may include a fist, a pointing finger, or other anatomical positions or poses.

Such joint angles between body parts or anatomical positions of body parts may more generally be referred to as a topography of such body parts. In general, a topography of body parts may refer to relative positions or orientations of the body parts. Further, as the embodiment shown illustrates, embodiments such as those described herein may infer, from deformation of one part of a body (the forearm 106 in the embodiment shown), one or more joint angles, one or more anatomical positions, or (more generally) a topography of one or more body parts (the hand 186 and fingers of the hand 186) that are not within a sensor (the sensor 102 in the embodiment shown) but that rather may be outside of (or spaced apart from) a part of the body (the forearm 106 in the embodiment shown) where deformation is measured and that may be movable relative to the part of the body where deformation is measured.

As another example, movement of an elbow adjacent the forearm 106, of one or more fingers of the hand 186, of a shoulder on a same arm as the forearm 106, or of still other body parts may be inferred from measurements of deformation of the forearm 106.

An anatomical position, or a sequence of anatomical positions at respective different times, stored in the anatomical positions store 240 may represent a gesture or a user input. Therefore, after block 238, the program codes 166 continue at block 242, which includes codes that, when executed by the microprocessor 152, cause the processor circuit 150 to determine whether an anatomical position, or a sequence of anatomical positions at respective different times, stored in the anatomical positions store 240 may represent a gesture or a user input.

An example of a sequence of anatomical positions at respective different times is illustrated in FIG. 14, which illustrates schematically a time series of deformation measurements 244 including a deformation measurement 246 associated with the hand 186 in a fist anatomical position, a deformation measurement 248 associated with the hand 186 in an anatomical position in which the index finger 188 is in the pointing position, and a deformation measurement 250 associated with the hand 186 in an open anatomical position.

If at block 242 an anatomical position, or a sequence of anatomical positions at respective different times, stored in the anatomical positions store 240 may represent a gesture or a user input, then the program codes 166 continue at block 252, which includes codes that, when executed by the microprocessor 152, cause the processor circuit 150 to store, in a gesture or user input store 254 in the storage memory 154, one or more codes representing the gesture or user input identified at block 242.

After block 252, or if at block 242 a gesture or user input is not identified, the program codes 166 continue at block 256, which includes codes that, when executed by the microprocessor 152, cause the processor circuit 150 to cause the output signal interface 162 to produce one or more output signals in response to respective positions of one or more underlying body parts stored in the position of underlying body part store 202, one or more joint angles stored in the joint angles store 224, one or more anatomical positions stored in the anatomical positions store 240, one or more gestures or user inputs stored in the gesture or user input store 254, or a combination of two or more thereof.

After block 256, the program codes 166 may return to block 168 as described above, so that measurements and inferences may be handled iteratively over a period of time.

Other inferences may be made. For example, speed, force, or both of movement may be detected or inferred, for example from one or more measurements or inferences of how forcefully or how fast a muscle contracts. Fit of the sensor 102 (or of another wearable or of other clothing) and volume of a muscle for a specific user may also be measured and inferred. Such measurements of inferences may indicate whether a size of a muscle changes over a period of time.

In general, the one or more output signals may control the display device 105 or one or more other display devices in different applications depending on the inferences such as those described above or calculations based on deformation measured by the sensor 102. For example, the one or more output signals may control the display device 105 in a gaming application, or the one or more output signals may control a virtual-reality, augmented-reality, or mixed-reality display. As another example, the one or more output signals may control one or more robotic devices. As another example, the one or more output signals may cause the display device 105 to display one or more anatomical positions stored in the anatomical positions store 240 at one or more different times, and such displays may facilitate analysis of body movements for analysis of sports performance, medical diagnosis, or other purposes. In alternative embodiments, program codes may cause the processor circuit 150 may predict gestures or user inputs based on specific muscle bundle or bone or tendon movement.

Also, in general, such control of the display device 105 may be real-time or may be delayed. For example, control of the display device 105 responsive to measurements of deformations by the sensor 102 may involve controlling a gaming application, a virtual-reality, augmented-reality, or mixed-reality display, or one or more robotic devices in real-time, or may display anatomical positions inferred from measurements of deformations by the sensor 102 in real time. Alternatively, such control of the display device 105 may be delayed. For example, anatomical positions inferred from measurements of deformations by the sensor 102 may be stored and accumulated over time, and may be displayed later.

In summary, in the embodiment described above, when the user moves fingers of the hand 186, the hand 186, or the forearm 106, deformation measurements by the deformation sensors may be used to form a time-dependent MTDT of the forearm, 106, which may represent movement (such as gradual movement, for example) of specific muscle bundles, bones, tendons, or two or more thereof within the forearm 106, and such movement can be related (in real time, for example) to movements (such as gradual movements, for example) of the hand 186 or of one or more fingers of the hand 186, including transitions between gestures.

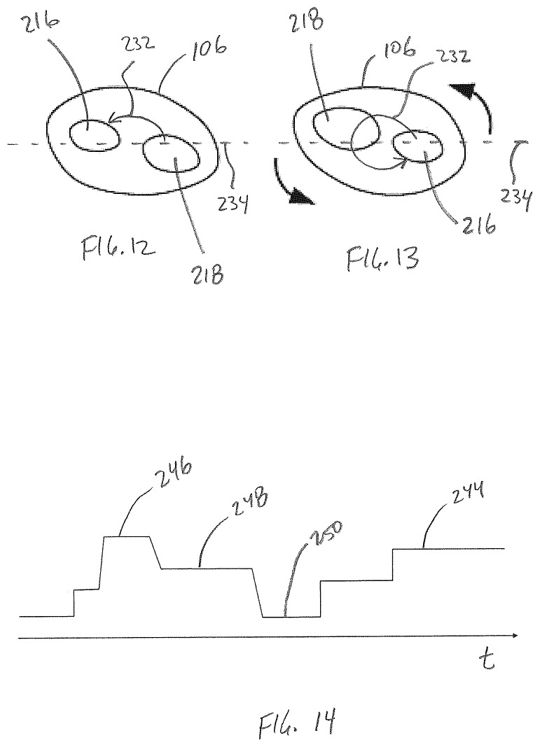

Referring to FIGS. 15-17, a sensor 258 according to another embodiment includes a resiliently deformable material sized to be received tightly on a lower leg 260 of a body, and configured to surround the lower leg 260. The sensor 258 also includes a plurality of deformation sensors, such as deformation sensors 262 and 264, for example, and the deformation sensors of the sensor 258 are positioned in the sensor 258 in a two-dimensional array and spaced apart from each other such that, when the sensor 258 is worn on the lower leg 260, the deformation sensors of the sensor 258 are spaced apart from each other in at least two directions and therefore form a grid or two-dimensional array. The sensor 258 also includes a data processing unit 266 that may function similarly to the data processing unit 120 as described above.

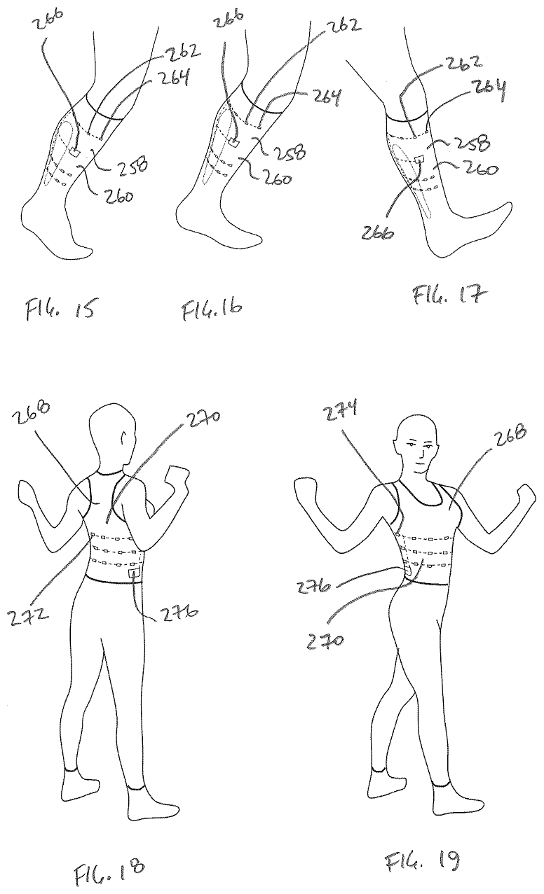

In this embodiment, the sensor 258 may provide MTDT monitoring for accurate detection and monitoring of walking patterns, gait, or running habits. Referring to FIGS. 15-17, the plurality of deformation sensors of the sensor 258 may cover the calf muscles (gastrocnemius, extensor digitorum longus, or tibialis anterior, for example), tendons, and fascia, which may facilitate measuring accurate and real-time MTDT from lower leg movements during different stages of walking and running, including a toe-off stage (shown in FIG. 15), a swing phase (shown in FIG. 16), and a heel strike (shown in FIG. 17), for example.

Although the sensor 258 is shown on a lower leg 260, sensors of other embodiments may sense movements of body parts, such as a thigh, a hip, one or more buttocks, or a combination of two or more thereof.

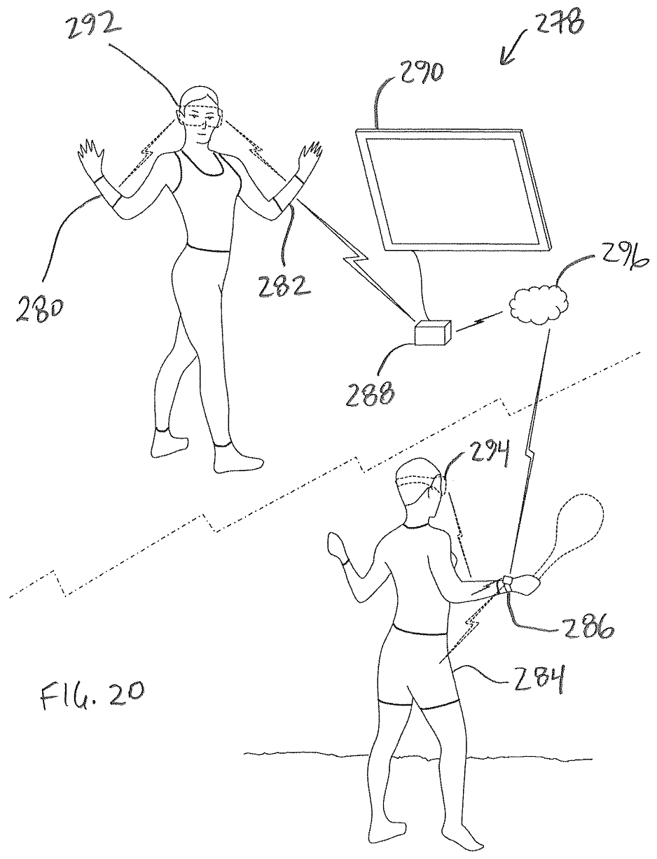

Referring to FIGS. 18 and 19, a sensor 268 according to another embodiment includes a resiliently deformable material sized to be received tightly on a torso 270 of a body, and configured to surround the torso 270. The sensor 268 also includes a plurality of deformation sensors, such as deformation sensors 272 and 274, for example, and the deformation sensors of the sensor 268 are positioned in the sensor 268 in a two-dimensional array and spaced apart from each other such that, when the sensor 268 is worn on the torso 270, the deformation sensors of the sensor 268 are spaced apart from each other in at least two directions and therefore form a grid or two-dimensional array. The sensor 268 also includes a data processing unit 276 that may function similarly to the data processing unit 120 as described above.

Accurate placement of the plurality of deformation sensors, such as deformation sensors 272 and 274, both anterior and posterior sides of the torso 270 (on a chest, abdomen, and back, for example), may enable measuring MTDT data from some or all of the upper body. The deformation sensors placed on the torso 270 (or for example the chest and epigastrium) may, in addition, measure respiratory rate, respiratory pattern, heart rate, heart rate variability, or other vital signs. The plurality of deformation sensors can measure MTDT from both the anterior and posterior side of the torso 270, which can be associated with body movement such as shoulder stretch and/or rotational movements of the torso 270.

Sensors of other embodiments may be in a shirt, a top, a vest, or other upper-body garments or wearables.

### Alternative Embodiments

The system 100 is an example only, and alternative embodiments may differ.

For example, referring to FIG. 20, a system for estimating a topography of at least two parts of a body is shown generally at 278 and includes sensors 280 and 282 on a first body, sensors 284 and 286 on a second body different from the first body, a computing device 288, and a display device (such as a television) 290, a display device (such as virtual-reality, augmented-reality, or mixed-reality goggles) 292 on the first body, and a display device (such as virtual-reality, augmented-reality, or mixed-reality goggles) 294 on the second body.

As shown in FIG. 20, the sensors 280 and 282 and the display device 292 may be in communication with each other using a wireless protocol, for example, and the sensors 284 and 286 and the display device 290 may be in communication with each other using a wireless protocol, for example. As also shown in FIG. 20, the computing device 288 and the sensor 286 may communicate with each other using a computer network (such as the Internet) 296.

In general, different embodiments may include multiple sensors on the same body, which may be in communication with each other, and which may facilitate measurements more accurately or more comprehensively than a single sensor. Further, one or more sensors on multiple bodies (as shown in FIG. 20, for example) may facilitate collaboration, game play, or other interaction. Such multiple bodies may be near each other (in a same room, for example) or remote from each other.

Further, multiple computing devices such as those described herein may execute the same or complementary programs, and may interact with each other using a computer network (such as the Internet, for example).

### Conclusion

In summary, sensors such as those described herein may be worn on one or more parts of a body, and may measure deformations that may be associated with movements of one or more other parts of the body. Such associations may provide input for applications such as virtual reality, augmented reality, mixed reality, robotic control, other human-computer interactions, health monitoring, rehabilitation, sports and wellness, or gaming, for example.

Although specific embodiments have been described and illustrated, such embodiments should be considered illustrative only and not as limiting the invention as construed according to the accompanying claims.

1-96. (canceled)

97. A method of estimating a topography of at least first and second parts of a body, the method comprising:
causing at least one processor circuit to receive, from a plurality of deformation sensors positioned on the body, at least one signal representing a plurality of measurements of deformation of at least a portion of the body, wherein the plurality of measurements of deformation comprise, for each deformation sensor of the plurality of deformation sensors, measurements of deformation of the deformation sensor at least along at least one direction along which the deformation sensor extends along the body;
causing the at least one processor circuit to associate the deformation with relative positions of at least the first and second parts of the body,
wherein causing the at least one processor circuit to associate the deformation with the relative positions of the first and second parts of the body comprises causing the at least one processor circuit to, at least, associate the plurality of measurements of deformation with a respective position of at least one underlying body part underlying the plurality of deformation sensors,
wherein the at least one underlying body part comprises at least one muscle, at least one bone, or at least one tendon, and
wherein the body is a human body or a non-human animal body; and
causing the at least one processor circuit to produce at least one output signal representing the relative positions of at least the first and second parts of the body; and
wherein each of the plurality of deformation sensors comprises:
a fiber mesh comprising a plurality of elongate fibers, wherein each fiber of the plurality of elongate fibers comprises an electrical conductor comprising an electrically conductive exterior surface reversibly positionable into and out of electrically conductive contact with the electrically conductive exterior surfaces of adjacent fibers of the plurality of elongate fibers; and
at least one resiliently deformable encapsulating film that encapsulates the fiber mesh, whereby resilient deformation of the at least one encapsulating film moves fibers of the plurality of elongate fibers and reversibly controls electrically conductive contact between the exterior surfaces of adjacent fibers of the plurality of elongate fibers and changes electrical resistance of the fiber mesh.

98. The method of claim 97 wherein the plurality of deformation sensors are spaced apart from each other in at least two directions, and the plurality of measurements of deformation are at locations spaced apart from each other in the at least two directions.

99. The method of claim 98 wherein the at least two directions comprise:
a direction along the body part underlying the plurality of deformation sensors; and
a direction around the body part underlying the plurality of deformation sensors.

100. The method of claim 98 wherein a sensor comprises the plurality of deformation sensors, and at least an entire portion of the sensor comprising the plurality of deformation sensors extends along each of the at least two directions.

101. A system for estimating a topography of at least first and second parts of a body, the system comprising:
a plurality of deformation sensors positionable on the body, wherein each deformation sensor of the plurality of deformation sensors is configured to measure deformation of the deformation sensor at least along at least one direction along which the deformation sensor extends along the body when the plurality of deformation sensors are positioned on the body;
at least one processor circuit configured to, at least:
receive, from the plurality of deformation sensors, at least one signal representing a plurality of measurements of deformation of at least a portion of the body, wherein the plurality of measurements of deformation comprise, for each deformation sensor of the plurality of deformation sensors, measurements of the deformation of the deformation sensor at least along the at least one direction along which the deformation sensor extends along the body when the plurality of deformation sensors are positioned on the body;
associate the deformation with relative positions of at least the first and second parts of the body by, at least, associating the plurality of measurements of deformation with a respective position of at least one underlying body part underlying the plurality of deformation sensors, wherein the at least one underlying body part comprises at least one muscle, at least one bone, or at least one tendon;
produce at least one output signal representing the relative positions of at least the first and second parts of the body; and
wherein each of the plurality of deformation sensors comprises:
a fiber mesh comprising a plurality of elongate fibers, wherein each fiber of the plurality of elongate fibers comprises an electrical conductor comprising an electrically conductive exterior surface reversibly positionable into and out of electrically conductive contact with the electrically conductive exterior surfaces of adjacent fibers of the plurality of elongate fibers; and
at least one resiliently deformable encapsulating film that encapsulates the fiber mesh, whereby resilient deformation of the at least one encapsulating film moves fibers of the plurality of elongate fibers and reversibly controls electrically conductive contact between the exterior surfaces of adjacent fibers of the plurality of elongate fibers and changes electrical resistance of the fiber mesh.

102. The system of claim 101 further comprising:
a sensor comprising the plurality of deformation sensors;
wherein the at least one processor circuit is further configured to associate the deformation with the relative positions of at least the first and second parts of the body by, at least:
inferring information indicating at least stretch of the sensor around the at least one underlying body part.

103. The system of claim 101 further comprising a sensor textile comprising the plurality of deformation sensors, wherein the sensor textile is wearable on the body, and wherein the sensor textile is stretchable and configured to hold the plurality of deformation sensors against at least the portion of the body.

104. The system of claim 101 wherein the first part of the body comprises the portion of the body.

105. The system of claim 101 wherein the at least one processor circuit is configured to associate the deformation with the relative positions of the first and second parts of the body by, at least, associating the deformation with relative positions of more than two parts of the body.

106. The system of claim 101 wherein the at least one processor circuit is configured to associate the deformation with the relative positions of the first and second parts of the body by, at least, associating the deformation with at least one joint angle, wherein the at least one processor circuit is configured to associate the deformation with the at least one joint angle by, at least, associating the deformation with the at least one joint angle in response to the respective position of the at least one underlying body part.

107. The system of claim 106 wherein the at least one joint angle comprises at least one angle of flexion or extension between the first and second parts of the body.

108. The system of claim 106 wherein the at least one joint angle comprises at least one angle of rotation between the first and second parts of the body.

109. The system of claim 106 wherein the at least one processor circuit is configured to associate the deformation with the relative positions of the first and second parts of the body by, at least, associating the deformation with at least one anatomical position of the first and second parts of the body, wherein the at least one processor circuit is configured to associate the deformation with the at least one anatomical position of the first and second parts of the body by, at least, associating the deformation with the at least one anatomical position of the first and second parts of the body in response to the at least one joint angle.

110. The system of claim 101 wherein the at least one processor circuit is configured to associate the deformation with the relative positions of the first and second parts of the body by, at least, associating the deformation with respective relative positions of the first and second parts of the body at a plurality of different times, wherein the at least one processor circuit is configured to associate the respective relative positions of the first and second parts of the body at the plurality of different times with at least one gesture or with at least one user input.

111. The system of claim 101 further comprising causing the at least one processor circuit to associate the relative positions of the first and second parts of the body with at least one anatomical position.

112. The system of claim 101 wherein:
the portion of the body comprises a forearm of an arm of the body, and the second part of the body comprises phalanges on the arm of the body;
the portion of the body comprises a lower leg of the body, and the second part of the body comprises a foot on the lower leg; or
the portion of the body comprises a torso of the body, and the second part of the body comprises the arm of the body.

113. The system of claim 101 wherein the at least one processor circuit is configured to produce the at least one output signal representing the relative positions of the first and second parts of the body by, at least, controlling at least one display in response to the relative positions of the first and second parts of the body, and wherein the at least one processor circuit is configured to control the at least one display in response to the relative positions of the first and second parts of the body by, at least, causing the at least one display to display at least one representation of the relative positions of the first and second parts of the body.

114. The system of claim 101 wherein the at least one processor circuit is configured to produce the at least one output signal representing the relative positions of the first and second parts of the body by, at least, controlling at least one robotic device in response to the relative positions of the first and second parts of the body.

115. The system of claim 101 wherein the at least one processor circuit is further configured to, at least, calibrate positions of the plurality of deformation sensors relative to anatomical features of a musculoskeletal model.

116. The system of claim 102 wherein the information indicates at least fit and stretch of the sensor around the at least one underlying body part.

117. The system of claim 101 further comprising stretchable wire lines between respective ones of the plurality of deformation sensors.

118. The system of claim 101 wherein the plurality of deformation sensors are stretchable.

119. The system of claim 100 wherein the at least one processor circuit is further configured to, at least, infer a volume of a muscle.

120. The system of claim 101 wherein the at least one processor circuit is configured to associate the deformation with the relative positions of the first and second parts of the body by, at least, associating the plurality of measurements of deformation with the relative positions of the first and second parts of the body according to a statistical learning algorithm trained to associate deformation of the portion of the body with the relative positions of the first and second parts of the body.

121. The system of claim 101 wherein the plurality of deformation sensors are spaced apart from each other in at least two directions, and the plurality of measurements of deformation are at locations spaced apart from each other in the at least two directions.

122. The system of claim 121 wherein the plurality of deformation sensors are configured such that, when the plurality of deformation sensors is positioned on the body, the at least two directions comprise:
a direction along the body part underlying the plurality of deformation sensors; and
a direction around the body part underlying the plurality of deformation sensors.

123. The system of claim 121 further comprising a sensor comprising the plurality of deformation sensors, wherein at least an entire portion of the sensor comprising the plurality of deformation sensors extends along each of the at least two directions.

124. The system of claim 101 wherein the second part of the body is spaced apart from and movable relative to the portion of the body.

125. The system of claim 101 further comprising a sensor comprising the plurality of deformation sensors, wherein the sensor is stretchable.

126. A system for estimating a topography of at least first and second parts of a body, the system comprising:
a plurality of deformation sensors positionable on the body;
at least one processor circuit configured to, at least:
receive, from the plurality of deformation sensors, at least one signal representing at least one measurement of deformation of at least a portion of the body;
associate the deformation with relative positions of at least the first and second parts of the body by, at least, associating the deformation with a respective position of at least one underlying body part underlying the plurality of deformation sensors, wherein the at least one underlying body part comprises at least one muscle, at least one bone, or at least one tendon, and wherein the second part of the body is spaced apart from and movable relative to the portion of the body; and
produce at least one output signal representing the relative positions of at least the first and second parts of the body;
a sensor textile comprising the plurality of deformation sensors; and
a furniture cover comprising the sensor textile or bedding comprising the sensor textile.
